(19) United States
(12) Patent Application Publication
(10) Pub. No.: US 2021/0265055 A1
(43) Pub. Date: Aug. 26, 2021

(54) Smart Meditation and Physiological System for the Cloud

(71) Applicants: Wilbert Q. Murdock, Bronx, NY (US); Philip Alister Williams, (US)

(72) Inventors: Wilbert Q. Murdock, Bronx, NY (US); Philip Alister Williams, Salt Point, NY (US)

(73) Assignee: Wilbert Quinc Murdock, Bronx, NY (US)

(21) Appl. No.: 17/235,988

(22) Filed: Apr. 21, 2021

Related U.S. Application Data
(63) Continuation-in-part of application No. 15/799,217, filed on Oct. 31, 2017.
Application No. 15/799,217 is a continuation-in-part of application No. 11/901,552, filed on Sep. 18, 2007, now Pat. No. 9,802,129.
Application No. 11/901,552 is a continuation-in-part of application No. 15/466,569, filed on Mar. 22, 2017, now Pat. No. 10,653,964.
Application No. 15/466,569 is a continuation-in-part of application No. 12/799,529, filed on Apr. 26, 2010, now Pat. No. 9,662,558.
(60) Provisional application No. 63/013,137, filed on Apr. 21, 2020.

(51) Int. Cl. G16H 40/67; A61B 5/16; A61B 5/375

(52) U.S. Cl. CPC G16H 40/67; A61B 5/375; A61B 5/168; A61B 5/165

(57) ABSTRACT

The invention incorporates a network and modules to register and monitor a group leader and a plurality of participants for a specific group activity, each remotely located from each other and a group physiological state server. Each of a plurality of module and individual registration information of each participant is stored in the form of data in memory on the server and individual participant data is stored at each participant's location. The second module monitors and processes real-time physiological measurements for each of the participants in the specific group activity. A third module tracks a physiological state of the participants, and fourth module evaluates and applies artificial intelligence to provide real-time feedback concerning the physiological state of the group or of an individual. A fifth module may also be employed to provide feedback pertaining to the group leader's physiological state in relation to the overall group physiological state.

The Smart Meditation and Physiological system invention is an advanced group meeting network where meditation brain waves, alpha-meditation and stress, beta-concentration and intellectual activity, gamma-focus and engagement, theta-attention span (such as if subscribers are drifting off to sleep during a session) are recorded and analyzed, yielding collective neurological information to guide the host, physician, instructor, or motivational speaker real-time feedback during a group session.

Moreover, the invention aids in evaluating and facilitating group meditation and yields emotional information of one or more individual participants to monitor the effectiveness of an individual and or group meeting, or a religious prayer session, as well as providing physiological information. It is also a health maintenance diagnostic system all-in-one that continually monitors the health of each individual user in real-time and provides an overall assessment of the effectiveness of the session, a media presentation, or a group meeting, and the information is easily accessible to the host and or to all parties within the group, if desired, housed in a cloud-based system. The information allows the instructor, clergy, or host to perfect and or curtail their next sermon, conference, class, or motivation talk to a particular audience.

### CROSS REFERENCE TO RELATED APPLICATIONS

This application is a utility application of provisional application Ser. No. 63/013,137, filed Apr. 21, 2020 and is a continuation of U.S. patent application Ser. No. 15/799,217, filed Oct. 31, 2017, which, in turn, is a continuation-in-part of U.S. patent application Ser. No. 11/901,552, filed Sep. 18, 2007, U.S. patent application Ser. No. 15/466,569, filed Mar. 22, 2017, U.S. patent application Ser. No. 12/799,529, filed Apr. 26, 2010, each of the above-referenced applications incorporated herein by reference as if restated in full.

### FIELD OF THE INVENTION

The invention relates to computer networking, but far more specifically to providing real-time feedback on collective physiological states of user groups engaged in activities where the users can be remotely located from each other.

### BACKGROUND

Typical prayer, conference, and or meeting systems over the Internet are problematic in that there is no feedback to leaders with respect to class participants. On Zoom and other video conference systems, although there is video and audio feedback, there are also many other activities that involve more than just video and audio.

For example, a group leader may be unable to tell how attentive students are in a meeting. A media advertisement is unable to ascertain the effectiveness of the advertisement. A prayer or meditation session may involve more than just what can be seen or heard. Further, a physician may need to examine a patient remotely via the Internet. Therefore, there is a need for a robust technique to provide real-time feedback on collective physiological states of user groups engaged in activities.

### SUMMARY

These shortcomings are addressed by the present disclosure of methods, computer program products, and systems for providing real-time feedback on collective physiological states of user groups engaged in activities.

In one embodiment, a system includes a processor, a network interface, communicatively coupled to the processor and the plurality of sensors over the data communication network; and a memory, communicatively stored to the processor. The memory stores a first registration module to register for monitoring a group leader and a plurality of students for a specific group activity, each remotely located from each other and the group physiological state server; a second module to track at least one physiological sensor taking real-time physiological measurements for each participant or student participating in the specific group activity; a third module to track a physiological state of the participants; a fourth module to apply artificial intelligence for providing real-time feedback concerning the physiological state of the overall group interaction of the participants, and a fifth module to track the group leader physiological state in relation to the group physiological state.

Advantageously, group leaders obtain real-time physiological feedback for online sessions of remote group participants.

### BRIEF DESCRIPTION OF THE DRAWINGS

In the following drawings, like reference numbers are used to refer to like elements. Although the following figures depict various examples of the invention, the invention is not limited to the examples depicted in the figures.

FIG. 1 is a high-level block diagrams illustrating a group physiological state system for providing real-time feedback on collective mind states of groups engaged in activities, according to one preferred embodiment.

FIG. 2 is a more detailed block diagram illustrating a group physiological state server, according to one embodiment.

FIG. 3 is a more detailed block diagram illustrating physiological monitors, according to one embodiment.

FIG. 4 is a high-level flow diagram illustrating a method for group physiological state monitoring, according to one preferred embodiment.

FIG. 5 is a more detailed flow chart of the step of providing real-time feedback on collective mind states of groups engaged in activities, according to one embodiment.

FIG. 6 is a block diagram illustrating an example computing environment for components of the system, according to an embodiment.

FIG. 7 is a high-level animated block diagram illustrating disease detection physiological and meditative state monitors, processed, and stored on the Cloud Server.

FIG. 8 is a high-level animated block diagram illustrating disease detection monitoring system related to corresponding parts of the anatomy.

FIG. 9 is a high-level diagram illustration of the steps pertaining to disease detection and progression monitoring stored on the server in a database 24/7.

FIG. 10 is a high-level animated diagram illustrating various physiological sensor devices to be monitored, data stored, processed with encryption, and evaluated using Artificial Intelligence.

FIG. 11 is a high-level animated diagram illustrating the steps from data acquisition of various activities to data encryption, data storage in a database and on the Cloud Server incorporating Artificial Intelligence.

FIG. 12 is another high-level animated diagram illustration of the steps of data acquisition of various activities to data encryption, data storage in a database and on the Cloud Server, incorporating Artificial Intelligence.

FIG. 13 is a high-level animated diagram illustrating various activities and or networks interacting with the network Flexible Subscription Service.

FIG. 14 is an animated diagram illustrating the facilitation and integration of prayer with the network and Cloud Server.

FIG. 15 is an animated diagram illustrating various activities, apparel, and or items interfacing with the network and Cloud Server.

### DETAILED DESCRIPTION

The description below provides methods, computer program products, and systems for providing real-time feedback on collective mind states of groups engaged in activities. The setting can involve guided meditation sessions, corporate boardroom meetings, group prayer sessions, teachers with online classrooms, and the like.

One of ordinary skill in the art will recognize many additional variations made possible by the succinct description of techniques below.

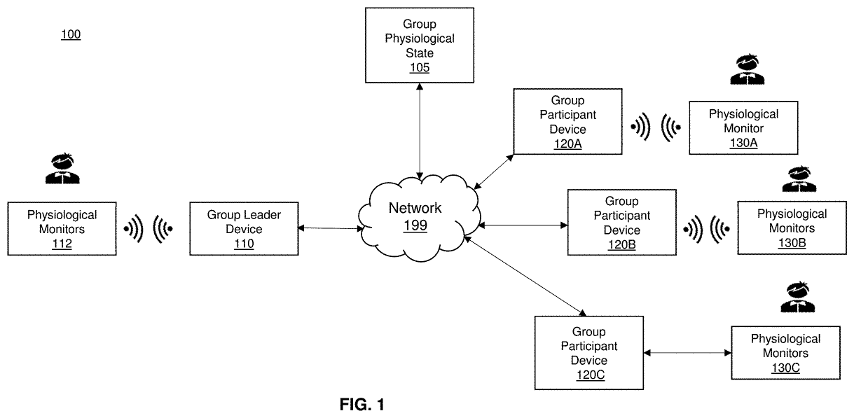

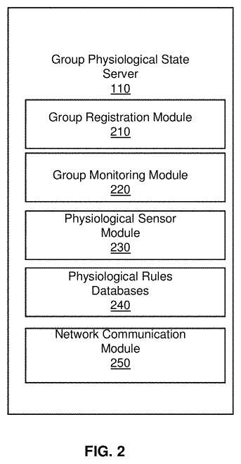

I. Systems for Group Physiological State Monitoring (FIG. 1-3)

FIG. 1 is a high-level block diagram illustrating a system 100 for a physiologic state system, according to one embodiment. The components of the system 100 are communicatively coupled through a network 199 which can be the Internet, a LAN, a WAN, a cellular data network (e.g., 3G, 4G or 5G), or the like.

The system 100 includes a group physiological state server 105 which is detailed in FIG. 2. Specifically, a group registration module 210 allows configuration of various groups participating in online sessions, including a group leader, group participants, and physiological parameters for the session. The parameters can be expected levels for the meeting, historical levels for past meetings, and other appropriate inputs. A group leader can schedule events, and participants can log into the group registration module during the meeting. In an embodiment, video conference software automatically configures groups for monitoring during sessions.

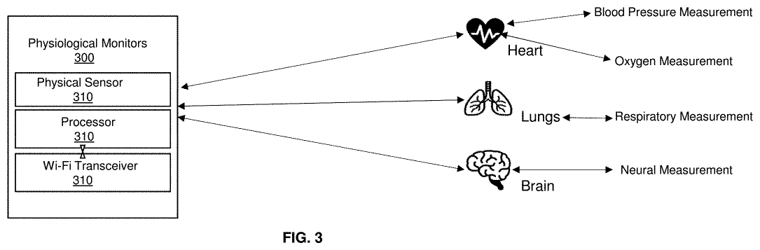

A group monitoring module 220 administers the meeting in real-time. To do so, input is received remotely from at a physiological sensor module 230 and is applied against rules from a physiological rules database 240. An example sensor shown in FIG. 3 receives inputs from individuals during a meeting or other type of session. The physiological monitor 300 is local to a participant and has physical sensors 310 attached to, for example, measure blood pressure, oxygen, respiratory system, and neurological aspects of the brain (e.g., alpha, beta and theta brain waves). Many other types of sensors are possible. A processor 320 calculates levels from the physical sensor 310 and uses a Wi-Fi transceiver 330 to send these levels upstream to the group monitoring module 220 of the group physiological state server 105. Moreover, a processor or equivalently a computer processor is hereon and heretofore understood to be, and or comprise, a microcontroller and or a microprocessor, and each of the latter is understood to be included in the former.

Finally, the network communication module 250 provides backend communication capabilities over a network 199. Application data is encapsulated in data packets formatted according to standards for Ethernet or Wi-Fi for transport. A transceiver places the information on a physical channel and receives communication from other components.

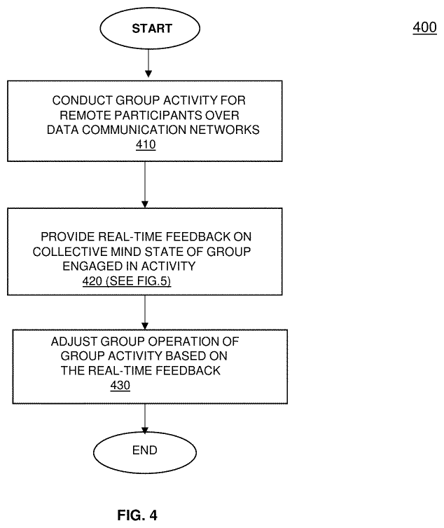

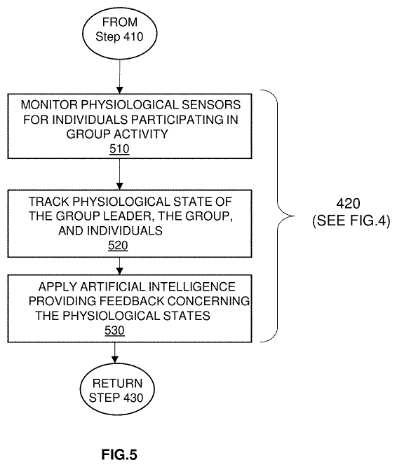

II. Methods for Group Physiological State Monitoring (FIGS. 4-5)

FIG. 4 is a high-level flow diagram illustrating a method for physiological state monitoring, according to one embodiment. The method 400 can be implemented, for example, by the system 100 of FIG. 1. The steps are merely representative groupings of functionality, as there can be more or fewer steps, and the steps can be performed in different orders. Many other variations of the method 400 are possible.

At step 410, group activities are conducted for remote participants over a data communication network. The group activity can be a prayer session, a meditation session, a company meeting, a remote classroom, or the like. At step 420, real-time feedback is provided on a collective mind state of the group engaged in the activity. At step 430, group operation is adjusted based on the real-time feedback. In particular, a group leader can change the agenda or switch prayer chants. A participant can get feedback on individual performance and make changes as well. One embodiment compares the group as a whole to the leader, to an individual student, or to an expected performance level.

During a group meditation session, for instance, a meditation leader can track when the collective group has entered beta wave stage when a threshold number of participants have reached a threshold brain wave measurement. In response, the meditation leader can change the type of guidance provided for the new collective brain state. Additionally, the group leader can be notified that a session will be ending and that the collective group should be guided from beta back to alpha wave state.

Returning to step 420 in more detail, at step 510 of FIG. 5, physiological sensors for individuals participating in the group activity are monitored. At step 520, a physiological state of the group leader, the collective group and/or individual participants is tracked. At step 530, rules are applied concerning physiological states. One embodiment applies artificial intelligence to determine actions, suggestions, and other feedback.

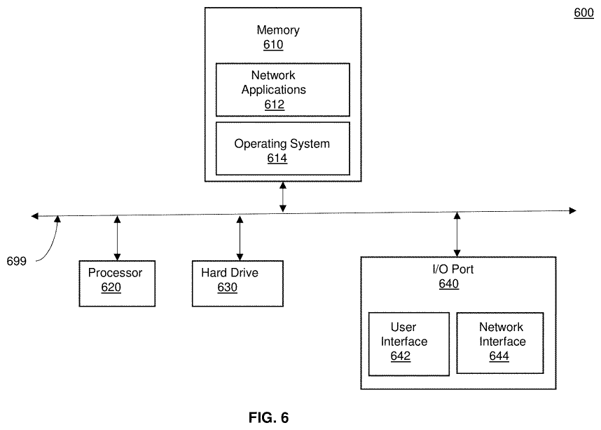

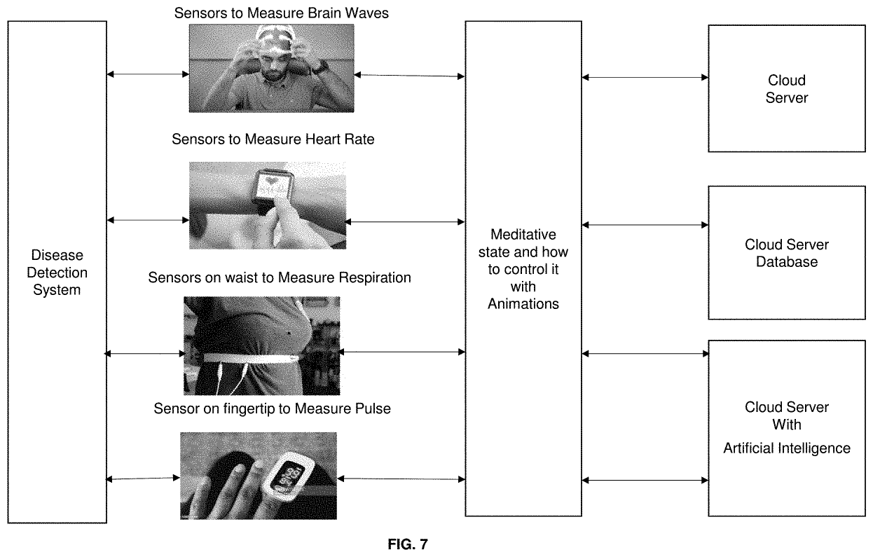

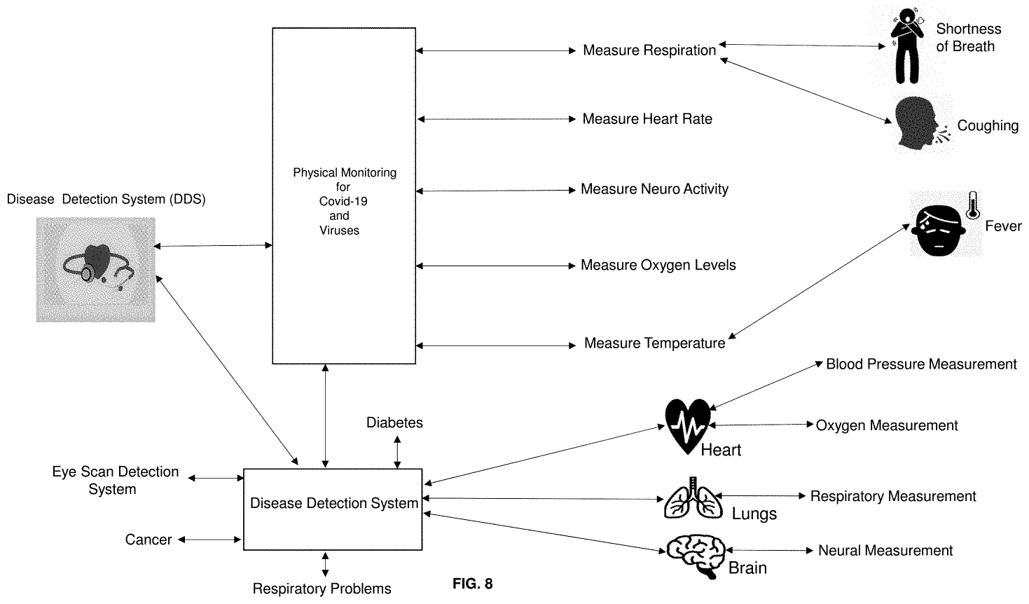

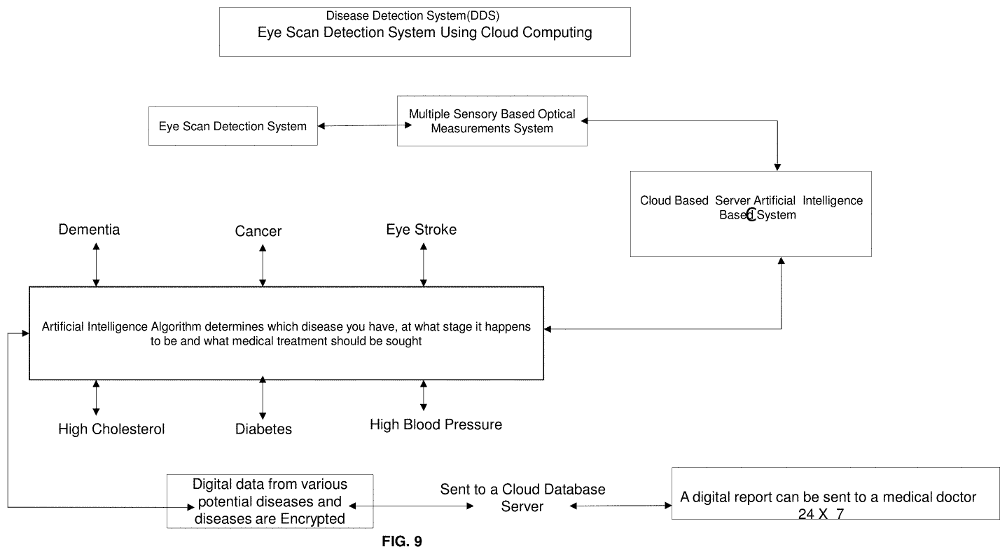

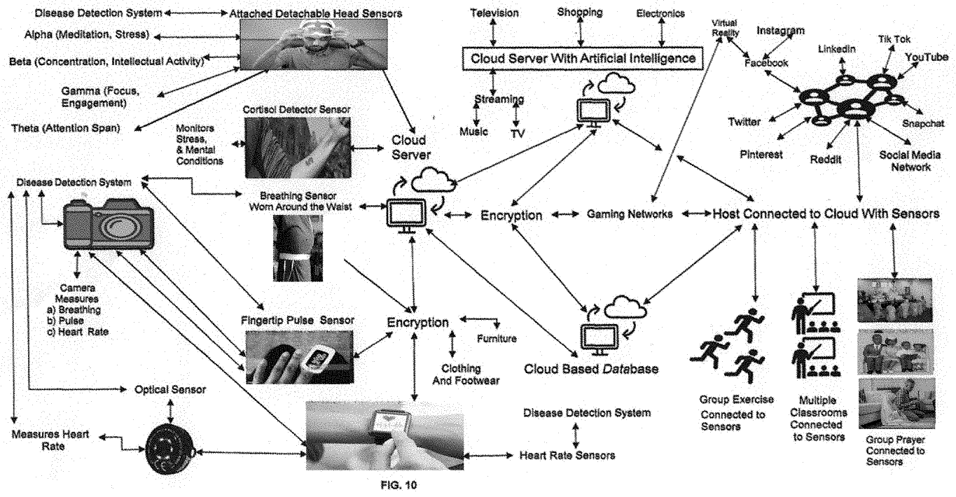

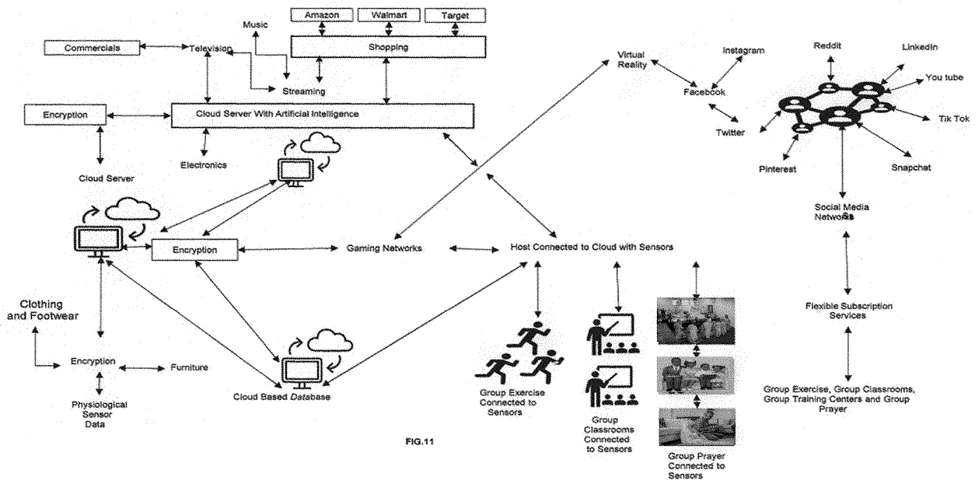

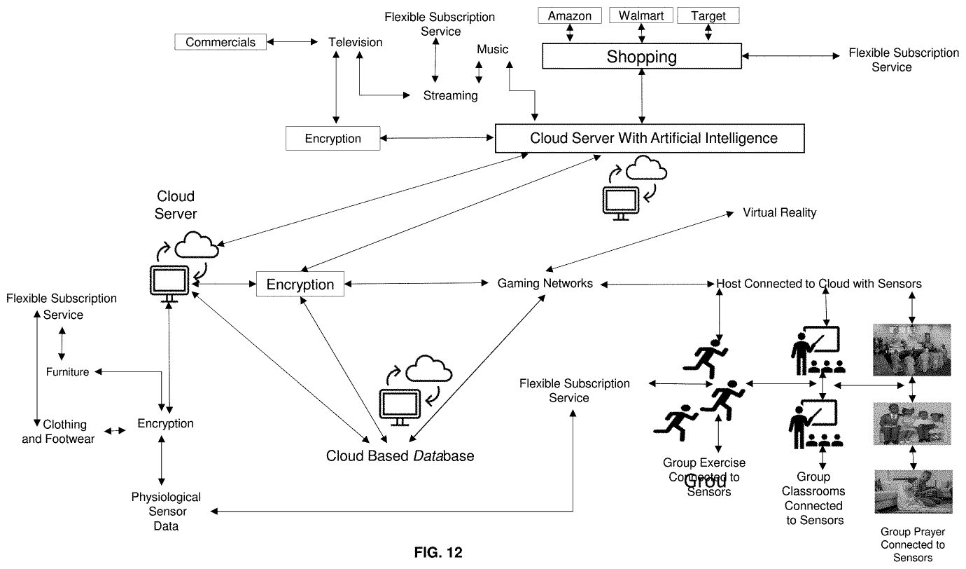

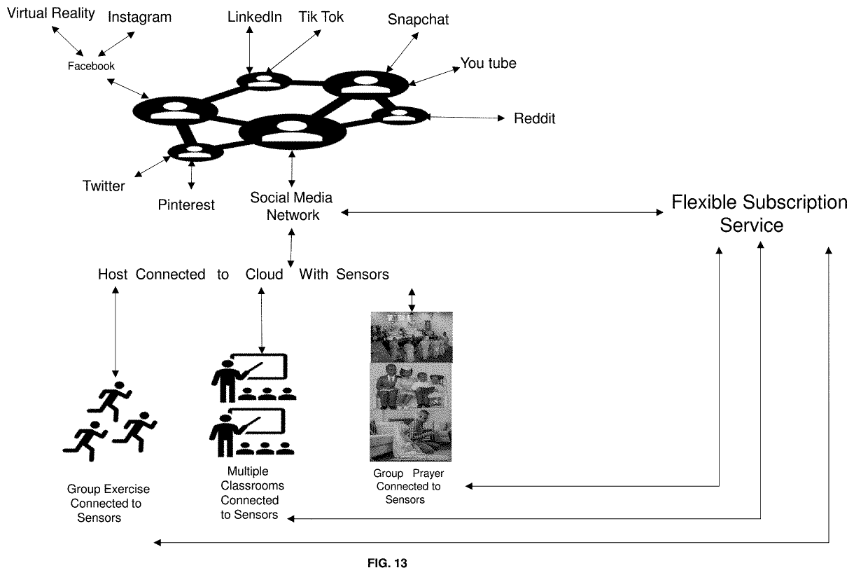

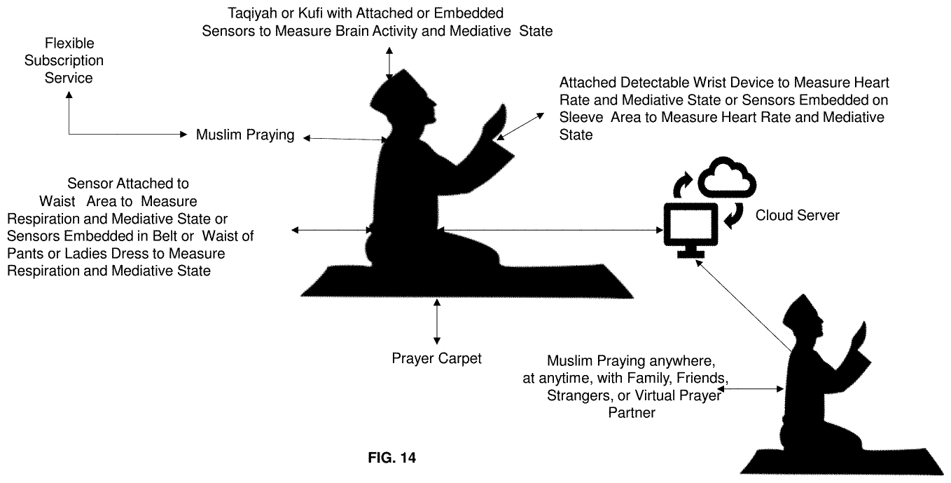

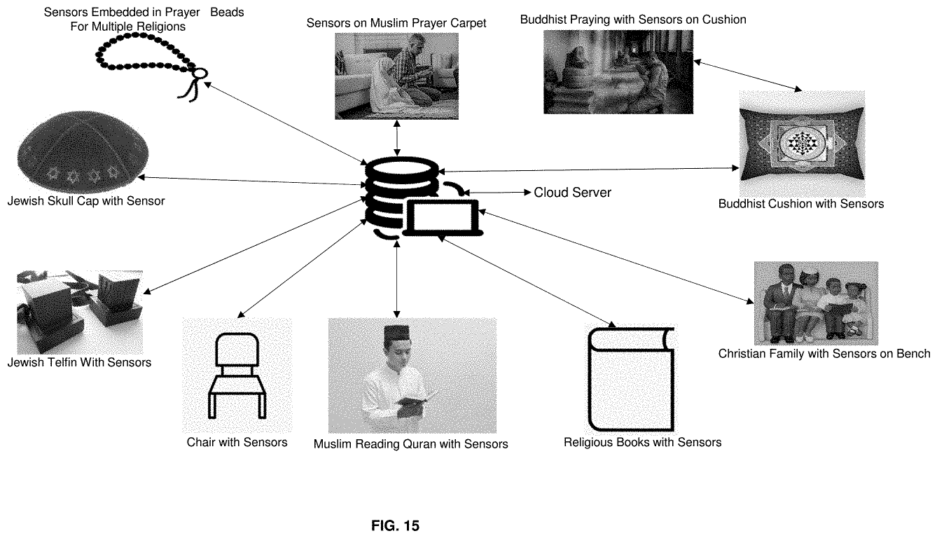

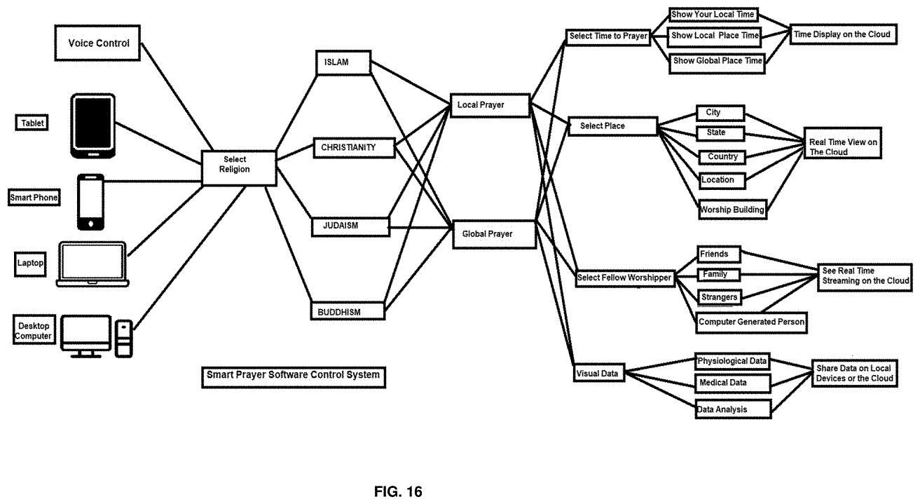

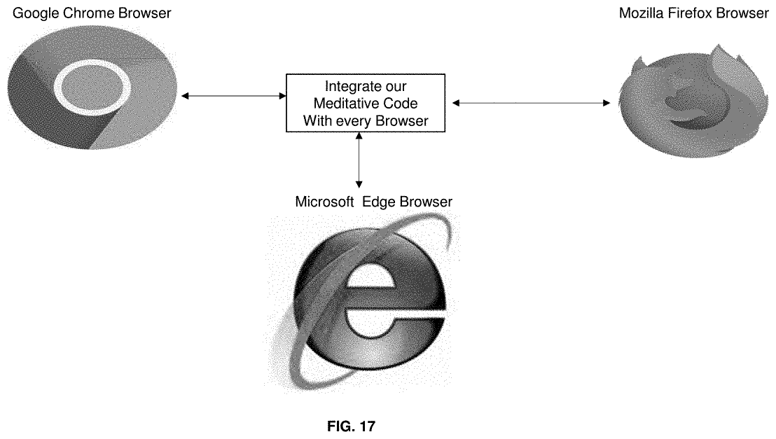

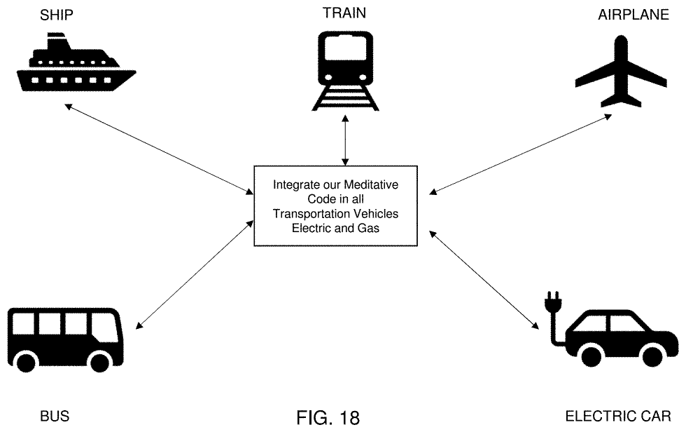

III. Generic Computing Device (FIG. 6)

FIG. 6 is a block diagram illustrating an example computing device 600 for use in the system 100 of FIG. 1, according to one embodiment. The computing device 600 is implementable for each of the components of the system 100. The computing device 600 can be an autonomous vehicle or a control system on an autonomous vehicle, a vehicle communication device, a mobile computing device, a laptop device, a smartphone, a tablet device, a phablet device, a video game console, a personal computing device, a stationary computing device, a server blade, an Internet appliance, a virtual computing device, a distributed computing device, a cloud-based computing device, or any appropriate processor-driven device.

The computing device 600 of the present embodiment, includes a memory 610, a processor 620, a storage drive 630, and an I/O port 640. Each of the components is coupled for electronic communication via a bus 699. Communication can be digital and/or analog and use any suitable protocol.

The memory 610 further comprises network applications 612 and an operating system 614. The network applications 612 can include a web browser, a mobile application, an application that uses networking, a remote application executing locally, a network protocol application, a network management application, a network routing application, or the like.

The operating system 614 can be one of the Microsoft Windows® family of operating systems (e.g., Windows 96, 98, Me, Windows NT, Windows 2000, Windows XP, Windows XP x64 Edition, Windows Vista, Windows CE, Windows Mobile, Windows 6 or Windows 8), Linux, HP-UX, UNIX, Sun OS, Solaris, Mac OS X, Alpha OS, AIX, IRIX32, IRIX64, or Android. Other operating systems may be used. Microsoft Windows is a trademark of Microsoft Corporation.

The processor 620 can be a network processor (e.g., optimized for IEEE 802.11, IEEE 802.11AC or IEEE 802.11AX), a general-purpose processor, an application-specific integrated circuit (ASIC), a field programmable gate array (FPGA), a reduced instruction set controller (RISC) processor, an integrated circuit, or the like. Qualcomm Atheros, Broadcom Corporation, and Marvell Semiconductors manufacture processors that are optimized for IEEE 802.11 devices. The processor 620 can be single core, multi-core, or include more than one processing elements. The processor 620 can be disposed on silicon or any other suitable material. The processor 620 can receive and execute instructions and data stored in the memory 610 or the storage drive 630.

The storage drive 630 can be any non-volatile type of storage such as a magnetic disc, EEPROM (electronically erasable programmable read-only memory), Flash, or the like. The storage drive 630 stores code and data for applications.

The I/O port 640 further comprises a user interface 642 and a network interface 644. The user interface 642 can output to a display device and receive input from, for example, a keyboard. The network interface 644 (e.g., RF antennae) connects to a medium such as Ethernet or Wi-Fi for data input and output.

Many of the functionalities described herein can be implemented with computer software, computer hardware, or a combination.

Computer software products (e.g., non-transitory computer products storing source code) may be written in any of various suitable programming languages, such as C, C++, C#, Oracle® Java, JavaScript, PHP, Python, Perl, Ruby, AJAX, and Adobe® Flash®. The computer software product may be an independent application with data input and data display modules. Alternatively, the computer software products may be classes that are instantiated as distributed objects. The computer software products may also be component software such as Java Beans (from Sun Microsystems) or Enterprise Java Beans (EJB from Sun Microsystems). Some embodiments can be implemented with artificial intelligence.

Furthermore, the computer that is running the previously mentioned computer software may be connected to a network and may interface with other computers using this network. The network may be on an intranet or the Internet, among others. The network may be a wired network (e.g., using copper), telephone network, packet network, an optical network (e.g., using optical fiber), or a wireless network, or any combination of these. For example, data and other information may be passed between the computer and components (or steps) of a system of the invention using a wireless network using a protocol such as Wi-Fi (IEEE standards 802.11, 802.11a, 802.11b, 802.11e, 802.11g, 802.11i, 802.11n, and 802.11ac, just to name a few examples). For example, signals from a computer may be transferred, at least in part, wirelessly to components or other computers.

In a further embodiment, with a Web browser executing on a computer workstation system, a user accesses a system on the World Wide Web (WWW) through a network such as the Internet. The Web browser is used to download web pages or other content in various formats including HTML, XML, text, PDF, and postscript, and may be used to upload information to other parts of the system. The Web browser may use uniform resource identifiers (URLs) to identify resources on the Web and hypertext transfer protocol (HTTP) in transferring files on the Web.

In the quest for creating technologies to monitor health and or be able to reprogram the body to achieve a new state of complete calm, which many have called meditation, there has yet to be an effective way to monitor these activities and quantify this activity except by someone stating they feel better. For few technologies or tools exist to quantity the meditation state, and constantly monitor in real-time its health benefits. For decades there has been a movement towards better health and being able to monitor states of health on a global scale but doing it one individual person at a time. So, beyond the mid 2000 era, we started to create technologies (Patent application 980212962—Internet Sports Computer Cellular Device) which were focused on an individual, but also took into account multiple individuals whose data profile could be captured, information processed, stored, shared, and made available on the Cloud.

One of such widely well-known technologies, which several companies lay claim to is the ability to measure heart rate on the wrist area with an attachable-detachable device. It is the one device which sparked the global revolution for health monitoring and maintenance on and off the Cloud. Our device even back then, data could be digitally projected to most surfaces without the need for the user to look at his or her wrist device. Obviously, the human heart is one of several major organs of the human body, which needs constant monitoring, along with the brain and lungs. If your heart beats an irregular pattern this could be a sign of heart problems or disease.

One of such widely well-known technologies, which several companies lay claim to is the ability to measure heart rate on the wrist area with an attachable-detachable device. It is the one device which sparked the global revolution for health monitoring and maintenance on and off the Cloud. Our device even back then, data could be digitally projected to most surfaces without the need for the user to look at his or her wrist device. Obviously, the human heart is one of several major organs of the human body, which needs constant monitoring, along with the brain and lungs. If your heart beats an irregular pattern this could be a sign of heart problems or disease.

This became a major game changer in terms of health and in health monitoring, since up until that time many people from around the world would have to go to their physician, whether it was a primary healthcare specialist or a cardiologist. Typically, these patients would be connected to a blood pressure machine or a medical specialist would use a stethoscope to listen to the heart. The next best thing to monitor a heartbeat or rate was using a Holter/Monitor, which gained more of a reputation as being too large to sleep with over a 24-hour cycle and not taking an accurate picture of the data a cardiologist is interested in. If a patient was not having a heart infarction or some type of fibrillation problem, the Holter/Monitor was useless. Unfortunately, heart disease and heart attacks do not usually occur at your Doctor's office. We figured out long before everyone else, that heart monitoring had to be done on a portable level. Portability, married to monitoring and measuring heart rate on the wrist area created a multi-billion-dollar revolution in healthcare that we originally started. Once we created the primary system to measure heart rate on the wrist area, it is extremely easy to determine different types of cardiac problems with changes to the software and refinement and upgrades to the hardware.

This signaled the advent and start of a portable disease detection data acquisition system combined with Cloud Computing. Our technology is now at the forefront of helping to solve an especially important problem in Covid-19 among other viruses now and in the future. Obviously, this is a global problem. One of the embodiments of this invention is to take physiological measurements. This includes measuring heart rate on the wrist area or placing a sensor on the heart or using a camera or optical sensor and measuring respiration with sensors placed on the waist area or lung area or throat area and measuring neurological activity with sensors placed on the head area, or sensors inside a hat or cap, and measuring oxygen levels with sensors placed on the fingertip or using a camera. By measuring the data from the 3 major organs of the body, the heart, the lungs, and the brain, we can better analyze the data and make a direct correlation between shortness of breath, unusual coughing patterns, and high fever to Covid-19 or some other virus.

By using multiple physiological data, the processor can further analyze the data and look specifically at detecting new illnesses such as cancer, diabetes, and respiratory problems with the aid of external peripheral medical tools. Another embodiment spawned from this invention is the eye scan detection system. A multiple sensory based optical measurement device focuses specifically on the eye region, the processor sends the data to an analog to digital converter, and the data is sent wirelessly to a first computer, and the data is then sent to a second computer or cloud-based server with built-in artificial intelligence algorithms.

The artificial intelligence algorithm determines and identifies what type of disease you may be developing, the current stage of the disease, and then suggests the potential treatments. The eye scan detection system is purported to look for diseases such as dementia, cancer, eye strokes, high cholesterol, diabetes, and high blood pressure. The digital data from dementia, cancer, eye strokes, high cholesterol, diabetes, and high blood pressure of an individual or multiple individuals, or a family of individuals, or a town of individuals, can be viewed in real-time in an encrypted state to hide the identity of the participants if they so desire it. Yet participants who give permission for the data to have a name associated for research studies to help avoid or lessen the impact of the next generation virus, then the information can be viewed in real-time to determine realistic patterns of potential problems.

This invention integrates cloud computing with sensors, cloud databases, and a cloud server with Artificial Intelligence, using a flexible subscription or registration system, with one or more central host or artificial intelligence hosts. Both clusters of data or information is sent to the Cloud Server Database and different forms of a digital report can be sent to a physician, government official, or user. Digital reports regarding your health care data pertaining to you as an individual or your entire family can be sent electronically to the Doctor, anywhere and anytime.

The principles of meditation are also combined with the measurement of physiological functions using sensors and sensory feedback and present it in a unique way using the Cloud. In patented application U.S. Pat. No. 9,802,129B2, we illustrated via graphics and talked about measuring physiological brain activities using a sensory feedback system, using sensors on the brain, capturing these data from one or more persons, and sending the data to the cloud. The one most important question is what meditation is. Some say it is the art of teaching people to better focus the mind and or redirect thoughts. Meditation is also associated with finding peace, balance, a sense of calm, which makes it good for overall health and well-being. Some suggest closing your eyes, breathe naturally, and focus your attention on a breathing pattern. Meditation slows down the flow of information to the brain, which insures that it does not get overwhelmed with too much information. This in turn ensures better processing of the information the brain must analyze. Meditation is the ultimate stress reducer for the brain and body that requires no medication or drugs.

There are several types of meditation, standing meditation, laying down meditation, and moving meditation among others, but the principles are the same for all different types of meditation. For many years the technology tools are and were virtually the same to gauge meditative activities. At the top of the list is the measurement of brain waves, using an EEG or Electroencephalogram. Using the EEG sensors attached or attachable-detachable sensors inside a hat, or cap, or religious headwear to the brain, small electrical activities can be measured and can also detect brain waves which act abnormally. Other more modern technology tools are the combination of EEG or brain measurement systems used independently or in combination with apps. Some apps may not use sensors wirelessly connected to the brain, but may instead concentrate on a person's breathing pattern, providing reminders and feedback on your individual breathing characteristics.

Measuring EEG is not enough information to give a person or group of people the complete data profile needed for understanding their own meditative state. It has remained more of an art than science, until now. When we measure an individual or group of participant's mediative state, our direct interest is filtering out alpha waves to see the progression of the meditative state and stress levels, and the beta waves to measure the level of concentration and intellectual capacity, and gamma waves to measure the level of focus and engagement and theta waves to measure attention span. Our system uses a cortisol detection sensor on the skin to measure and monitor stress levels and mental conditions.

In patent application U.S. Pat. No. 9,802,129B2, the graphics showed, and we talked about using sensors on the waist area to measure respiration. The system can have sensors embedded in a belt or the waist area of a pants or shirt to measure respiration. Both respiration measurement systems measure breathing, providing graphical and or animated feedback. A camera can also be used to measure respiration. Respiration rates are sometimes measured by a piezoelectric respiration sensor, which is worn around the chest area or by using infrared systems to measure chest compression and or movement in the diaphragm area. There may be a direct correlation between being able to see your respiration rate via animation and actively being able to control it during a meditative session.

This is currently the only known innovative to measure breathing and can inform a user or group of people on and off the Cloud if they are reaching the proper meditative state, and or are breathing properly. They can see a graphical or animated image of their specific breathing pattern, along with brain measurement activity via animation. Both the brain and breathing of said person or group of people, must be measured simultaneously and displayed via animation.

In patent application U.S. Pat. No. 9,802,129B2, it showed in the graphics and we spoke about using sensors on the wrist area to measure heart rate. The system can have sensors embedded on the sleeves of a garment, or sportswear, or religious outfit. Both heart rate measurement systems provide graphical and or animated feedback. A camera can be used to measure heart rate as well as an optical sensor system. All three physiological functions, i.e., brain, breathing, and heart measurements using sensors of said person and group of people, must be measured simultaneously and displayed via animation on and off the Cloud and the digital data can be projected onto a surface.

This invention is the use of a fingertip pulse sensor using a sensor on the fingertip area. A pulse can also be measured on the ear lobe and wrist area using a sensor. A camera can be used to measure a pulse as well. There may also be a direct correlation between being able to see your pulse rate via animation and actively being able to control it during a meditative session. Often pulse readings are measured to determine heart rate by attaching an electrode to a fingertip or ear lobe or reading one's pulse in the hands.

Physiological measurements for heart rate, brain waves, including implantable sensors, and or attachable sensors, and or detachable sensors, to help diagnose pulse, respiration rates, and chemical changes on the skin, are sent to the multifunction physiological processor wirelessly for analysis and processing. The digital data from the brain, respiration, heart, pulse, and skin are encrypted before sent to the Cloud. We wanted this technology to be the complete picture, of how to measure the meditative state and be a disease detection system at the same time.

Physiological measurements have now been expanded to a biosensor, ultrasound sensor, accelerometer sensor, lidar sensor, sonar sensor, video camera sensor including video streaming, piezo sensor including electric and resistive, eye sensor, infrared sensor, capacitive, hand sensor, tilt sensor, system on a chip sensor, electric field sensor, foot pressure sensor, nano or mini computerized tomography sensor, magnetometer sensor or nano-magnetometer sensor, graphene sensor, resistive sensor, fingerprint sensor or nano-fingerprint sensor, pedometer sensor, blood glucose sensor or nano-blood glucose sensor, pulse oximeter sensor or nano-pulse oximeter sensor, MRI sensor or nano-MRI sensor, GSR or skin moisture sensor or nano-GSR sensor, real-time location sensor or nano-real-time location sensor, gyro-meter sensor or nano-gyro-meter sensor, gyroscope sensor or nano-gyroscope sensor, magnetic compass sensor or nano-compass sensor, hand sensor or nano-based-hand sensor, white space sensor or nano-white space sensor, etc. The physiological information set of heart-rate data, respiration data, brain wave data, and sweat data are wirelessly sent to the physiological processor simultaneously.

The multifunction physiological processor wirelessly secures, processes, and analyzes heart rate, respiration rate, brain waves and many other physiological functions, simultaneously on and off the Internet or Cloud using a variety of well-known and established technologies. Among these technologies are the electroencephalogram for measuring brain waves to tell the differences between the alpha and beta states whereby sensors are attached to the head area. However, heart rates are measured by an electrocardiogram. A multifunction physiological processor and the multifunction sound processor work in a similar fashion.

The stored data on the electronic system console is physiological and motion data from the absolute best meditators locally and globally. The physiological data stored may comprise heart rate measurements, respiration rate measurements, and brain wave activity measurements, among others.

The method of storing these data further comprising the steps of gathering of physiological data by a multifunction processor and selecting specific physiological data, transmission of the selected physiological data to a second multifunction processor, selecting further specific physiological data, transmission of the selected physiological data to a third multifunction processor, filtering of specific physiological data, transmission of physiological data for electronic system console storage of physiological data, and comparing the user physiological data with previously stored physiological data on and off the Internet or Cloud.

The physiological data is at least one of heart-rate data, respiration data and brain wave data wirelessly sent to the physiological processor simultaneously. Other physiological body data are available in this system. The previously stored physiological data may be physiological data relating to professional athletes. The physiological system has wireless input/output interfaces, intelligent receivers, with the proper logic circuit to determine which physiological data should be sent first to processor, second processor, and third processor, A/D conversion for heart rate data, respiration data, brain wave data, to filter the heart rate data, respiration data, filter the brain wave data, and a processing means to analyze and interpret a plurality of received wireless physiological data.

Group Exercise on the Cloud

An embodiment of this invention is having one or more host on the cloud, that could measure a group or groups of people exercising anywhere or anytime also on the cloud, and can see their meditative levels in real-time, which exercises work, and or concentration techniques that work, or words of encouragement that may work. It can provide the ultimate encouragement and a direct visual guide through animation for faster improvements to all members within the group. The host could be a human or an artificial intelligence (A.I.) host. In either case, the exercise group on the cloud can speak Spanish, Chinese, Arabic, German, or Italian, etc., and if the human or A.I. host speaks English, the exercise group language would be translated automatically to English. If the human host or A.I. host spoke Chinese and the exercise group on the cloud spoke English, the host or A.I. host language would be translated to English. If the human host or A.I. host spoke English and the multiple exercise groups spoke multiple languages that is not English, each group would receive instructions in his or her native language.

The exercise groups on the cloud, have optional language choices for the voice instructional coming from the host or artificial intelligent host. Each individual or group exercise optimum meditative levels on the cloud are measured in a dynamic state and or a static state.

The Classroom or Board Room or Office Room or Theatre

Another part of this invention is having one or more host on the cloud, who could measure a group or groups of people in a classroom or board room or office setting or theatre anywhere anytime also on the cloud, and can see their mediative levels, in real-time, what words are working, or concentration techniques are working, or what visual and breathing exercises are working. In also provides the ultimate in encouragement and a direct visual guide through animation for immediate improvement to all members within the group.

The host could be a human, or an artificial intelligence host. In either case, the classroom group on the cloud can speak Spanish, or Chinese or Arabic or German, or Italian, etc. and if the host speaks English or A.I. host speaks English, the classroom group language would be translated automatically to English. If the human host or AI host spoke Chinese and the classroom group on the cloud spoke English, the host or artificial intelligence. host language would be translated to English. If the human host or artificial intelligence host spoke English and the multiple classroom or board room or office room groups spoke multiple languages, that is not English, each group would receive instructions in his or her native language choice.

The exercise groups on the cloud have optional language choices for the voice instruction coming from the host or artificial intelligence host. Each individual user or group classroom optimum meditation levels on the cloud are measured in a static state or in a dynamic state like group exercise.

Religious Prayer

Another part of this invention is having one or more host on the cloud that could measure a group or groups of people in a prayer session anywhere, anytime, also on the cloud, and they can view their mediative levels in real-time, what religious words and prayers invoke the best levels or concentration techniques, and or what visual and breathing exercises work best, before, during, and after prayer.

The host could be a human or an artificial intelligence host. In either case, the prayer group on the cloud can speak Spanish, Chinese, Arabic, German, or Italian, etc., and if the human host or A.I. host speaks English, then the prayer session group language would be translated automatically to English. If the human host or A.I. host spoke Chinese and the prayer group on the cloud spoke English, the host or artificial intelligence host language would be translated to English. If the human host or artificial intelligence host spoke English and multiple prayer groups spoke multiple languages, that are not English, each group would receive instructions in his or her native language choice.

The prayer groups on the cloud have optional language choices for the voice instructions coming from the host or artificial intelligence host. Each individual or group prayer optimum meditative levels on the cloud are measured in a static state or dynamic state.

Group Prayer Electronic System

This invention relates to a system coupling actual meditative devices with smart prayer-like carpets or horizontal surfaces, prayer beads, religious prayer shawls and or tzitzit, benches, chairs and or sitting surfaces, religious clothing (i.e., Thobe and or Kasaya), religious hats (i.e., Kufi and or Yarmulke, Hijab and or Scarf and or Church Hat and or Habit and or Tefillin) and adaptive optics, a processor, a multi-core processor, computer, quantum computer, tablet, digital television, smart cell phone, and or portable communication devices, digital devices, multiple wireless protocols including cellular and 5G, digital compass, digital camera, artificial intelligence camera, laser light, scanning laser, fingerprint sensor, photoplethysmography sensor, pulse oximeter, electroencephalography, nanorobots, the use of holograms, digital image projector, artificial intelligence, bitcoins, blockchain, 3D images, digital visualizations and cloud based databases, cloud based AI sensory fusion, cloud based physiological health monitoring systems, in real-time chat.

Most worshippers, if they cannot make it to their place of worship, may be just lucky to experience the day's sermon via streaming technologies, which is linear by nature, and affords virtually zero interactivity, but is considered to be state-of-the-art. Streaming religious ceremonies is totally influenced by the worshiper's imagination as well as that person's meditative state. If a worshipper is incredibly young, sick, handicapped or elderly, getting to their favorite religious house of worship becomes problematic and tension filled, if not impossible. Women with large families or who work constantly, or men who work constantly, or families who work constantly, may not be able to get to their favorite place of worship because of having terribly busy schedules, which builds up a lot of tension.

Usually it is the larger mosques, churches, synagogues, or temples, which can afford to stream their services, hourly, daily, weekly, monthly, yearly. Streaming is limited and cannot tell the host or religious teacher the meditative or health state his or her parishioners are in. Currently streaming only provides one-way communication from the religious leaders to their flock. Language and time barriers limit each religious streaming session to that of the native-born worshipper, yet each religion is global, and narrows its appeal and technological flexibility. Our new system eliminates the one-dimensional nature of streaming used for religious worship, but also adds 2 new dimensions, which are the ability to measure a prayer group meditative state and monitor their health state as well. Our system captures a considerably basic need to know about your place of worship intimately. Human beings are very demanding, and always want to have the real experience as if they were physically present at worship, experiencing the same sensory feedback as their fellow worshipers, experiencing the same feelings, excitement, and emotions as their fellow worshippers, physically present in the house of worship. This now becomes a major technological revolution for religious worship.

This invention now makes the impossible possible by giving each Muslim, each Christian, each Jew, each Buddhist, each Hindu, the full ability to participate and experience every religious ceremony anytime, anyplace, locally, and globally on the Internet or Cloud, with multiple levels of sensory feedback including measuring a religious group meditative and health state. With the smart prayer system, now worshipers, now have the ability to participate at Hajj for Muslims in real-time or pray at the Prophet's Mosque in Medina for Muslims, or pray at the Wailing Wall in Jerusalem for Jews, or pray at the Mahabodhi Temple in India for Buddhists, or pray at the Vatican for Christians, or pray at Ayodhya (one of the seven holy cities) for Hindus, but also have a new technology tool to measure the group's health state as well as their meditative state.

Using Artificial Intelligence (AI), and Cloud based AI, combined with Cloud Based Databases, we will be able to identify what type of meditative data it is, where the meditative data is coming from in terms of the region of the world, the size of the meditative data sets, language translation, worshiper profile, what the meditative data means, managing the meditative data, exchanging meditative data, posting meditative data, and storing meditative data and retrieving meditative data. Religious worshipers locally and globally now have a new Cloud based network to enable them to pray with one another locally and or remotely with family, friends, and fellow worshipers. For worshipers to know the physical status i.e., health status and meditative state of other worshipers, especially family and close friends, the physical or health status must be captured, displayed, and stored in one or more databases on the cloud.

Social Media Networks

Social media networks like Facebook, LinkedIn, Tik-Tok, twitter, snapchat, reddit, Instagram, YouTube, Pinterest, and any new social media platform, have several things in common, groups of people socialize together sharing data and information in real-time and non-real time. Many times, people come together on social networks who know each other in business, or in a social setting like with family, or friends, some of which could be strangers. There is an obvious exchange of information, which can benefit an individual or the group.

Subjects as diverse as politics, cooking, exercise, meditation, medicine, covid-19 and or pandemics, favorite movies, television programs, and music are often discussed. Facebook is a social media network, which allows family and friends to connect and share various types of content. Some social media network such as Linkedin brings together businesspeople locally, nationally, and internationally to exchange business ideas or make business contacts or find new business partners not constrained by your business location and could also benefit from this technology.

Other social media platforms such as Youtube and Tik Tok share videos. Youtube videos allowed video creators to produce content of various lengths of time (minutes to hours) on a variety of topics allowing global audiences to access those videos anywhere, and anytime, on any digital platform.

What none of these social media platforms, have is the ability to know how their content, is directly affecting the customer or group of customers, and being able to measure that effect scientifically, with our meditative sensory system. By integrating our sensory based meditation network with any social media platform, it takes every system to the next generation. It also allows content creators to design better content and can attract more subscribers. to these various social media platforms. In fact, the content can be tested in real-time, before being uploaded. Our sensory based meditation system can be integrated in every social media platform with our code being a permanent feature on Facebook, YouTube, Snap chat, Tik Tok, LinkedIn, etc. or a downloadable app or a link on a social media platform to our technology.

Gaming Networks

Games by its very nature are fun but fun does not equate to being healthy. More gamers could be more attractive to every gaming network when our technology is integrated into every network and has real health benefits. This allows the creation of an entirely new generation of games, which are fun and have health benefits. For gaming network, our code could be integrated with the network itself, and could be integrated with the gaming software as well.

Shopping Network

When a shopper goes online at Amazon, Walmart, Target, or any retailer, sometimes the shopping exercise can be fun and exciting. Conversely the shopping experience can be boring, and non-exciting. Several things can influence a fun shopping experience, and or easy shopping experience, how easy is a product to find, how a product looks, the description of the products, customer reviews of the product, its availability, and proposed delivery date. Besides using big data and analytics to study shopping habits, demographics of shoppers (which includes race, earning power or salaries, zip codes, various economic scores), product creation, the selection process of products, the manufacturing process of products, the distribution of products and the process, this is the state-of-the-art of the total shopping experience.

Americans shop sixty-nine (69%) percent online and twenty-five (25%) shop online per month. Billions of people shop online daily with trillions made per day. The shopping experience also may be subject to multiple focus groups, and some members of that focus group can be subject to being connected to a sensory feedback system but is usually done person-by-person.

To date, no one has looked at the real-time physiological data from shopping and or shoppers, which are included in our specific technologies of measuring meditative states on the cloud. Those meditative states via a sensory feedback gives the most important data in real-time ever created about, shopping habits, and products (old and new).

Our meditative technology can be integrated into the product itself, so as soon as a buyer clicks on the product, Amazons, or Walmart, or Target, or any shopping platform, a good or bad physiological response is immediately known to the retailer. By using our meditative technology flexible algorithms, an alert can be sent to a retailer that a product description is not clear, or the price is too high, or because a popular product is out of stock it can give a buyer a negative feeling about that same product. Our meditative technology flexible algorithms can make real-time changes in all aspects of the products, which would move in more in line with positive meditative feedback, resulting in an immediate increase in sales.

Clothing & Shoes

For years, clothing and shoes, have had sensors, attached, or embedded or woven, measuring pressure or forces on the entire foot area, and flexible strips that touch your skin on clothing, measure temperature, or use an accelerometer to measure a wearers movement, heart rate and or breathing, and the data is sent to a processor wirelessly. Measuring forces of the foot allows a person to be either retrained to walk better or receive physical therapy to strengthen a part of the lower extremity to improve a person's gait or making a better design for shoes.

Sensor-based clothing at that time was the entry point to telemedicine. Sensors on and inside clothing is decades old, but at no time, has anyone created a sensor-based meditation system for clothing and shoes. Our meditative sensory system for clothes can tell the user if a garment is healthy to wear or if it's not according to the animation image with respect to his or her body, like the color, form, feel and texture of the garment. This is an entirely new dimension of analyzing clothing, which has never existed before. Our meditative sensory system for shoes can tell the user if that shoe is healthy to wear or not, according to the dynamic animated image, indicating his or her body, like the color, comfort, style is something the body might like.

Furniture

In the last decade sensors, started being used in or was embedded inside furniture. For the most part, sensors are used for remembering comfortable positions of the body, let's say for a chair. Yet meditative sensory systems have never been used in or embedded in furniture for real-time determination of its health benefits. This opens the door for an entirely new generation of furniture.

Streaming Media

Streaming media is a way to view video and audio through a method, over the Internet. So, music and television programs are streamed over the internet on various independent channels, giving viewers options. For streaming music and video commercials (streaming commercials), are still a fact of life, for both. Many streaming media like Netflix, with about 204 million, is one of the biggest video streaming platforms worldwide, streams videos from movies and television shows and documentaries. Youtube is another global video streaming media system with about 2.3 billion members, and well as Disney plus another major streaming platforms, with about 95 million members. Our meditative sensory system technology can be integrated into either an audio or video streaming service, no matter the platform. Our meditative sensory computer code can be integrated on the server side or client side. Our meditative sensory computer code could be also integrated into every browser.

Flexible Subscription Service

Our flexible subscription service, with a built-in artificial intelligence elastic pricing model, adjustable to the economic realities of a specific country and standard of living. Lower prices for poor or modest countries, and higher prices for more wealthy countries, all determined by machine learning and artificial intelligence algorithms and interface.

Body Alignment Sensor Device

A body alignment device employing a plurality of sensors which are positioned about various areas of a user's joints and the data generated is on the Cloud or Internet via a Server. Pre and Post Alignment Data is on the Cloud. The system includes split-screen display capability displayed via computer code and televised demonstrations on Personal Computers, whereas now on handheld devices with spatial orientation capability using a gyroscope with multifunction motion sensors connected to the player and a multifunction motion sensing device, all containing circuits and contact or multifunction motion sensors coupled with signal processing and radio frequency wireless transmitter circuitry with other wireless protocols, thereby wirelessly communicate game performance and multiple physiological data and multiple body alignment data and multiple body posture data and motion and sports apparatus, gaming tool or sports implement information or data, location data using GPS device and gyroscope to a remote receiver-computer. The computer displays player information, human body motion information or data, multiple types of physiological information or data simultaneously and visually simulates and controls two meditative sensory users via the internet having similar equipment and remotely located from each other providing a graphical blueprint to learn. A real time human motion analysis system used on and off the Internet or Cloud incorporates infrared markers which as secured at various joints of the subject's body. The subject is caused to perform a motion as walking or running, while performing the motion, the subject is televised by means of a video camera. The video signal from the camera is processed to develop a line count and a horizontal position count based on the operation of a pixel counter. The presence of a marker is determined by the illumination from the marker above a background illumination. A comparator activates a series of latches which cause the latches to provide data in terms of an x and y coordinates which data is sent to a computer to indicate the location of the marker with respect to the video frame. In this manner the system tracks the markers as placed on the joints of a subject and produces the X and Y coordinate which is used by the computer to analyze the motion. Standard sports equipment may be retrofitted with the sensors and associated circuitry to convert such equipment into “smart equipment” for use with the system. The system controls game play and communicates game information data, motion data, location data, and multiple physiological information.

This invention relates to monitoring the force at joints of the human body simultaneously in real time on the Cloud or Internet with the caveat being able to look at any joint individually such as the knee, elbow, shoulder, etc. As can be ascertained, a great deal of joint injuries is suffered by athletes and various other individuals. Sometimes athletes injure multiple joints at the same time. Healthy body joints should be analyzed via X-rays, Cat-Scans, MRI's, and other diagnostic tools used to measure the motion of joints in real time with the data generated throughout the active life cycle of the athlete and stored on the Internet or Cloud and would be considered pre-injury data. When an injury takes place or post injury data, this too is stored on the Internet or Cloud and compared to the Pre-injury data, taking into consideration a person's height, weight, and sport. A computer algorithm analyzes the two sets of data, i.e. pre and post injury day to communicate with the sensors on each joint what to change and when to change it during an athletic event or competitive game.

A body alignment device employing a plurality of sensors which are positioned about various areas of a user's joints. The sensors are coupled to circuitry including a microprocessor, with accelerometers and gyroscopes, with optimum values indicating the correct body motion stored on the server during the exercise. These values are compared with the actual values being generated by a user in performing the same exercise, and if the actual values exceed those stored on the Internet or Cloud, the user is warned of the dangerous pending condition and to modify the exercise being performed.

This invention relates to a system that interconnects sensor and GPS sensory circuitry, gyroscopes, real smart sports equipment, and athletic human motion and swing and location data to a computer, and the Internet or Cloud and multiple physiological readings simultaneously off and on the Internet or Cloud. The use of multiple types of sound off and on the Internet or Cloud and the use of multiple types of wireless protocols off and on the Internet or Cloud to improve athletic performance and competition. In a preferred embodiment the computer is coupled wirelessly to a user, smart sports equipment or cloned smart sports equipment, a receptacle, or a sport-specific motion and GPS sensing circuitry component, and multiples streams of physiological and location data. Furthermore, this invention, with the components summarized below, allows one or more players to enter a competition against each other. Each player asks the computer who is available to play a contest. Once a player pairs up against another player anywhere in the world and play ensues, the computer and display show each participant's score via animation or graphics that preferably relate to a player's individual performance statistics. A single player may play without an opponent to practice and improve basic sport-specific skills using the computer and display to track performance. In an advantageous embodiment the present invention accomplishes this through a continuous process wherein the present invention establishes a plane of space, reads how the player moves his or her body within said plane of space, and determines the optimized levels of performance based on the player's age, body type, playing style, and years of experience, which are automatically inputted into the system from a variety of swing and motion data and location data points. All of this information can be transferred to a server by a client, minimizing the required storage of information on the unit system itself. The system also analyzes and corrects in real-time via 3D graphics and holograms joint alignment and posture.

A multifunction motion sensing device contains an array of uniformly distributed sensing transducers upon or proximate to the device surface. This multifunction motion sensing device may be a camera. In a preferred embodiment, the surface is a clam-shaped handheld device. The camera produces detectable varying characteristics representing the velocity, angle, and proximity of a user and or a piece sporting equipment relative to the surface of the device or frame view of the camera or infrared device. The variances are electronically processed into digitally coded information and remotely transmitted by an electrical communication circuit contained within or electronically connected to the device.

At each remote player site, wireless radio frequency equipment and or other multiple wireless protocols equipment receives the digitally coded transmitted signals from the user and or a piece of sporting equipment, a ball receptacle, and the multifunction motion sensing device and multiple physiological sensing devices in a singular physiological processor. The signals are demodulated and processed into serial binary data suitable for communications to the computer via either serial or parallel ports.

The meditative sensory system tool has a plurality of embedded and attachable detachable contact sensors and internal electronic circuitry including wireless protocols for on and off the Cloud such radio frequency transmitter, Zigbee, RFID, Bluetooth, Wi-Fi, Wi-Max, UMB, Sigfox, Thread, 2G (GSM), 3 G and 4 G, 5G, BLE, LTE Cato 0 1 & 3, LTE-M1, NB-IOT, Zwave, LoReWan, Ingenu, Weighless-N, Weightless-P, Weightless-W, ANT & ANT+, DigiMesh, Wi-Fi-ah, MiWi, EnOcean, Dash7, WirelessHART, 6LoWPan, White Space. Cellular and GPS Sensing Circuitry. At least one of the sensors is located at or proximate to optimal location on a meditative sensory tool face for contact. A wireless radio frequency transmitting circuit receives the serial digital data from the microprocessor and wirelessly transmits the information via an internal antenna to the receiver for subsequent processing by the computer.

Wireless Signal Receiver and Computer

At each player site, a wireless radio frequency signal receiver is connected to the computer by either the serial (USB) or parallel computer ports. The wireless signal receiver detects digitally coded radio frequency transmissions from the communication circuit associated with any of a meditative sensory equipment. The received transmissions are demodulated by the RF receiver circuitry connected to a microprocessor, which converts the demodulated data signal to serial binary coded data suitable for communications to a computer. The computer, under the control of the internally installed meditative sensory software program, monitors and directs the flow of communications between remotely located players via the internet and displays the simulations and performance information. In appropriate installations the wireless electromagnetic signals that communicate with the receiver may be infrared communications. At each remote player site, the computer under the control of the meditative sensory software system program monitors and controls initialization and the sequential play of the game, or alternatively, the individual player practice session. Upon start up by a player at a particular site, the system input parameters are set, and the system Internet and player data port interfaces are initialized as indicated. For Internet communications, the serial port of the computer is enabled in the preferred embodiment. A local player event listener is initialized. The main operational software (program) thread is run, and the system awaits data input from the appropriate computer communications port.

If the competitive play mode has been selected, the program generates a player participation request and sends the request to the internet server. Upon identification of a player opponent by the server, the program initiates the player identification sequence and sequential play begins. This software sequence and control routine occurs at each remote site where play has been initiated. During the game play sequences, the program generates the appropriate animation, display, and audio data and commands, and communicates with the associated display and speaker devices. Upon the occurrence of a local player event, the main operating program displays the event and communicates the event by causing a device transmission to be sent via the internet GGC server which displays the event for the opposing players and alerts an opposing player that it is his/her turn to play. The local player event maybe but is not limited to the physiological data readings, athletic human motion capture and analysis, sound feedback data, location data, body and posture alignment. The program contains time delay limits for player action, and delays of play beyond these limits generate play quit and disconnect signals. The event at also has the effect of indicating at that it is no longer the local player's turn and enables the socket event listener to detect an event from the remote player, again via the Internet.

If the single player practice mode is selected, the internet communications sequences are disabled, other software sequential operating routines continue as above described and the player's movement, multiple physiological data, human motion analysis and or meditative sensory GPS circuit and gyroscope information are communicated only to the computer located at the player's site and the performance information analyzed and displayed only at the local player's site. When a game is won, lost, or terminated, the software system generates the appropriate output signals, displays the player performance information, and resets to initial pre-game conditions. If one player opponent quits the game or is “timed out” (due to excessive delay in play) and the remaining player wishes to continue play, the software resumes an Internet search for another opponent.

The motion sensing device contains a multifunctional wireless processor which has built-in multiple protocols such as Radio Frequency, Bluetooth, Zigbee, Wi-Fi, Wi-Max, UWB, Cellular which detects and distinguishes various wireless protocols, for example Zigbee, RFID, Bluetooth, Wi-Fi, Wi-Max, UMB, Sigfox, Thread, 2G (GSM), 3 G and 4 G, 5G, BLE, LTE Cato 0 1 & 3, LTE-M1, Zwave, LoReWan, Ingenu, Weighless-N, Weightless-P, Weightless-W, ANT & ANT+, DigiMesh, Wi-Fi-ah, MiWi, EnOcean, Dash7, WirelessHART, 6LoWPan, White Space, Cellular and GPS Sensing Circuitry from each other. The multifunction wireless processor allows the present invention to use embedded single and multiplayer meditative sensory software to communicate and exchange information with other meditative sensory system, human motion processors, multifunction physiological processors, multifunction sound processors, multifunction alignment processor, multifunction posture processor, location data, multifunction attachable detachable drones embodying the present invention. The multifunction wireless processor can memorize whatever wireless protocol it last read to display information. The display can show animations specific to the wireless protocol and can project a digital image and holographic image.

Human Motion Processor

Human motion processor data from the internet sports computer cellular device can be posted on the client to the server or broadcasted on a peer-to-peer network. The present invention makes use of infrared markers or light emitting diodes. In said embodiment, the marker is a five-sided facet. The side facets slope from the main facet at an angle between 10 to 15 degrees about the front side of the facet. The LED device emits light upon activation of the same by a typical power supply, which may be a battery or other device. The power supply is also secured to the body of the user by conventional techniques. One can analyze the motion of an arm, a back and other body parts and develop the three dimensional (3D) or X, Y, and Z coordinate information for various body parts. For example, to measure the angle between the hip joint, one would know said coordinates at the knee joint with relation to the hip.

Stored coordinate information data from the human motion processor determines angular relationships between said athlete body joints as monitored in real-time inside of the internet sports computer cellular device to use computer algorithms to generate real-time stick figure generation or animation display, and to display real-time athletic motion from the internet sports computer cellular device to a client; to a cellular device to the server, and store real-time athletic motion from internet sports computer cellular device to the server.

A sports gaming system, handheld device wherein the handheld device opens into two halves. One half of the open handheld device comprises a plurality of buttons wherein said buttons are flat so that the handheld device can close completely. One half of the open handheld device comprises at least one camera capable of encoding digital images and videos digitally storing images and videos and stabilizing images and videos on and off the Internet or Cloud. The sports gaming system handheld device further comprises a plurality of custom processors that measure and store physiological data, device specific data, and user motion data in real-time, with at least one multifunction processor capable of transmitting physiological real-time data, device real-time data, and user motion real-time data, an electronic system console, at least one processor capable of transmitting videos and digital images in real-time to an electronic system console. Moreover, at least one multifunction processor receives real-time data from an electronic system console. At least one multifunction processor has built-in global positioning sensing capability system and motion sensor capability. At least one multifunction processor can transmit multiple motion data to a on a single or multi-screen monitor display. In an embodiment, the plurality of multifunction processors are comprising sensors that are attached wirelessly to the user, a central processing unit, a processor capable of storing general data, a multifunction processor capable of storing user physiological data and motion data, a multifunction processor capable of receiving multiple wireless transmissions simultaneously, a processor capable of detecting, storing, and receiving user data, a processor capable of determining strength and type of wireless connectivity and choosing the strongest most available protocol connection, a processor capable of connecting to at least one cellular device, a processor capable of password and informational storage, a processor capable of comparing data from differing users, a processor capable of monitoring multiple physiological data, a processor capable of monitoring motion data wherein motion data relates to sports actions performed by the user, a processor capable of monitoring handheld device system specific data on and off the Internet or Cloud.

The plurality of multifunction processors comprises a processor capable of receiving transmissions from the at least one electronic system console wherein said this transmission produces sound feedback related to at least one of the physiological real-time data streams, device real-time data and user motion real-time data, said sound feedback comprises at least one of: voice feedback, pre-programed feedback, audio downloads, satellite radio, Dolby sound, and Yamaha sound. Additionally, a processor capable of gathering physiological real-time data and stores data and motion real-time and stored data is stored and comparing it to stored data on the at least one electronic system console, said processor transmits differences from real-time and stored user data and stored data to the at least one electronic system console. The system device may also be capable of receiving and transmitting phone calls to other sports gaming system devices, other phones and cellular devices. The Sports Computer Cellular Device can be used to call other Mega Machines by speaking into a built-in microphone that processors your voice and uses it as a input mechanism and translates voice using an a to d converter and translates it into an internal dialing command The Mega Machine can call landlines (POTS), VOID, and Cellular Devices. The user can simply say a person name, and the Mega Machine would dial it, or say a specific telephone number, and the Mega Machine automatically dials it.

A method for a sports gaming system device, handheld device comprises providing the systems discussed herein, wherein a handheld device automatically detects wireless networks and automatically connects to a detected frequency. In another embodiment of the method, a set of preferences can be used to connect to a preferred wireless network. The processors may detect at least one but is not limited to the following frequencies: Zigbee, Bluetooth, RF, wi-fi Wi-Fi, wi-max Wi-Max, uwb UWB, local area network and cellular router.

Wireless Processor

The wireless processor receives, stores, analyzes, and processes a plurality of wireless protocols in processor, which can transfer physiological data for daily, weekly, and monthly comparisons to server. The connecting wirelessly a plurality of internet sports computer cellular devices to the client, broadcasting wirelessly a plurality of internet sports computer cellular device data using a peer-to-peer network, transferring wirelessly a player's data in a graphics or iconic form to another player remotely, for a side-by-side statistical comparison. Wireless processor searches for available wireless protocols.

Sound Processor

The sound processor built-into the internet sports computer cellular device reads, processes, converts, interprets, and stores voice and sound information represented by iconic maps telling the athlete whether he or she has good movement, bad motion, or needs improvement within the normal limits of optimize movement dynamics usually demonstrated by professional athletes and computer models based on individual input parameters such as height, weight, body type, skill level, body conditioning, mental alertness.

Real-time GPS motion data and Real-time GPS location data for the internet sports computer cellular device can receive real-time motion and location data from the original and cloned sports apparatus, or gaming tool, or sports implement and athletic swing mechanics because of a built-in digital camera which can be commanded to take a series of shots, one after the other, and be reconstructed via iconic maps from a computer algorithm, or capture and analyze streaming media from a digital camcorder. Similarly, the GPS device built into the original and cloned sports apparatus provides the internet sports computer cellular device with motion and location data.

Body Alignment Processor

Body Alignment Processor allows a means to receive, store, analyze and process a plurality of sensor data connected to body joints in real-time into a processor which can transfer alignment data for daily, weekly, and monthly comparisons to the server and or Cloud. Then connecting a plurality of internet sports computer cellular devices wirelessly to the client, broadcasting wirelessly a plurality of internet sports computer cellular device alignment data using a peer-to-peer network, transferring wirelessly a player's body alignment data remotely.

Sensor Fusion

With sensor fusion for body alignment anatomical parts, we can look at these multiple streams of data in real-time or dynamically simultaneously to determine which joints are going to be injured during game play or during a sport allowing for immediate correction. It opens the door for new insights into the human body in real-time using 3D models and or holograms whereby this new data is now stored on the Cloud for present and future comparisons for joint alignment problems and solutions. With sensor fusion for posture alignment anatomical parts, we can look at these multiple streams of data in real-time or dynamically simultaneously to determine which joints are going to create bad form and or posture during a game or a sports event allowing immediate correction. It opens the door for new insights into the human body in real-time using 3D models and or holograms, whereby this new data is now stored on the Cloud for present and future comparisons for posture alignment problems and solutions.

Body Alignment Sensor Monitoring Apparatus

Apparatus for monitoring the proper alignment of all body joints of users, comprising transducer or sensors means coupled to the body joints of said user and operative to provide an output signal indicative of the relative forces on the body joints during an exercise and or gaming, a memory having stored therein data indicative of proper force levels for said body joints during the exercise or game, a comparison means operative to compare the stored data on the Cloud with said output signal to provide an indication when said output signal exceeds said stored data on the Cloud signal, and responsive to said indication to provide a warning to said user according to the generation of said indication.

Posture Sensor Monitoring Apparatus

Apparatus for monitoring the proper posture of all body joints of users, comprising a transducer or sensor coupled to the body joints of said user and operative to provide an output signal indicative of the relative forces on the body joints during an exercise, a memory having stored therein data indicative of proper force levels for said body joints during said exercise, a comparison means operative to compare said stored data on the Cloud with said output signal to provide an indication when said output signal exceeds said stored data on the Cloud signal, responsive to said indication to provide a warning to said user according to the generation of said indication.

Posture Alignment Processor

Posture Alignment Processor allows means to receive, store, analyze, and process a plurality of sensor data connected to body posture in real-time into a processor which can transfer alignment data for daily, weekly, and monthly comparisons to server and or Cloud. Then connecting wirelessly a plurality of internet sports computer cellular devices to the client, broadcasting wirelessly a plurality of internet sports computer cellular device alignment data using a peer-to-peer network, transferring wirelessly a player's body posture data remotely.

This description of the invention has been presented for the purposes of illustration and description. It is not intended to be exhaustive or to limit the invention to the precise form described and many modifications and variations are possible in light of the teachings above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following enclosed claims.

1. A group physiological state server, coupled to a plurality of sensors over a data communication network, for providing real-time feedback on collective physiological states of user groups engaged in activities, the server comprising:
a processor;
a network interface, communicatively coupled to the processor and the plurality of sensors over the data communication network; and
a memory, communicatively stored to the processor and storing:
a first module to register and monitor a plurality of participants for a specific group activity, each remotely located from each other and the group physiological state server;
a second module to track at least one physiological sensor taking real-time physiological measurements for each of the participants in the specific group activity;
a third module to track a physiological state of the participants; and
a fourth module to apply artificial intelligence for providing feedback concerning the physiological state of the user group.

2. The group physiological state server of claim 1, wherein the specific group activity comprises an internet conference, wherein a first sensor comprises a respiration sensor and a second sensor comprises a brain wave sensor.

3. The group physiological state server of claim 1, wherein the specific group activity comprises an internet conference, wherein a third sensor comprises a heart rate sensor and a fourth sensor comprises a blood pressure sensor.

4. The group physiological state server of claim 1, wherein the fourth module determines whether the group is collectively within the desired state based on the sensor data being within a predetermined range.

5. The group physiological state server of claim 1, wherein the fourth module determines that the group has collectively exited a desired state based on the first and second sensor data.

6. The group physiological state server of claim 1, wherein the fourth module provides an alert to the group leader concerning misalignment of the group physiological state.

7. The group physiological state server of claim 1, wherein the fourth module compares the group physiological state to a desired physiological baseline based on the specific activity.

8. The group physiological state server of claim 1, wherein the physiological sensor is physically attached directly and or indirectly to the body of an individual person or a participant.

9. The group physiological state server of claim 1, wherein the physiological state is based on measurements from at least one physiological sensor.

10. The group physiological state server of claim 1, wherein the physiological state comprises a mental state defined by brainwave activity.

11. A group physiological state server, coupled to a plurality of sensors over a data communication network, for providing real-time feedback on collective mind states of user groups engaged in activities, the server comprising:
a processor;
a network interface, communicatively coupled to the processor and the plurality of sensors over the data communication network; and
a memory, communicatively stored to the processor and storing:
a first module to register and monitor a plurality of participants for a specific group activity, each remotely located from each other and the group physiological state server;
a second module to track at least one physiological sensor taking real-time physiological measurements for each of the participants in the specific group activity;
a third module to track a physiological state of the participants; and
a fourth module to apply artificial intelligence for providing feedback concerning the physiological state in relation to the group physiological state.
a fourth module to apply artificial intelligence for providing feedback concerning the physiological state of the user group.

12. The group physiological state server of claim 11, wherein the specific group activity comprises a media advertisement event, wherein a first sensor comprises a neuro-feedback brain wave sensor and a second sensor comprises a physiological sensor.

13. The group physiological state server of claim 11, wherein the specific group activity comprises a meditation or a prayer, wherein a second sensor comprises a physiological sensor, and a third sensor comprises a temperature sensor.

14. The group physiological state of claim 12, wherein the neuro-feedback brain wave sensor acquires data relating to the group and or a participant's attention span.

15. The group physiological state server of claim 11, wherein the fourth module determines whether the group is collectively within the desired state based on the sensor data being within a predetermined range.

16. The group physiological state server of claim 11, wherein the fourth module compares the group physiological state to a desired physiological baseline based on the specific activity.

17. The group physiological state server of claim 11, wherein the physiological sensor is physically attached directly and or indirectly to the body of an individual person or a participant.

18. The group physiological state server of claim 11, wherein the physiological state is based on measurements from at least one physiological sensor.

19. The group physiological state server of claim 11, wherein the physiological state comprises a mental state defined by brainwave activity.

20. The group physiological state server of claim 11, wherein the physiological state comprises a mental state defined by a respiratory rate and or an oxygen saturation level.
